SEQUENCE LISTING (1 sequence)

SEQ ID NO: 1 - DNA, 30 bp, Synthetic construct
  source 1..30: mol_type = other DNA
actggtcacctggtcatcctgccgtaactg

(19) United States
(12) Patent Application Publication
(10) Pub. No.: US 2025/0002940 A1
(43) Pub. Date: Jan. 2, 2025

(54) SYSTEMS AND METHODS OF MICROWELL PLATE ARRAY FOR LOCALIZED ELECTROPORATION

(71) Applicant: Northwestern University, Evanston, IL (US)

(72) Inventors: Horacio Dante Espinosa, Evanston, IL (US); Cesar Andres Patino, Evanston, IL (US); Prithvijit Mukherjee, Evanston, IL (US)

(21) Appl. No.: 18/752,184

(22) Filed: Jun. 24, 2024

Related U.S. Application Data
(60) Provisional application No. 63/511,331, filed on Jun. 30, 2023.

(51) Int. Cl. C12N 15/87; C12M 1/12; C12M 1/34; C12M 1/42; C12N 15/85

(52) U.S. Cl. CPC C12N 15/87; C12M 25/04; C12M 35/02; C12M 41/46; C12N 15/85; C12N 2800/107

(57) ABSTRACT

An electroporation device comprising a first circuit board including a first plurality of electrodes; a plurality of wells; and a bottomless well plate. Each of the plurality of wells at least partially received within the bottomless well plate. The electroporation device further includes a second circuit board including a second plurality of electrodes. The bottomless well plate is positioned between the first circuit board and the second circuit board. Each of the first plurality of electrodes is at least partially positioned within one of the plurality of wells.

### CROSS-REFERENCE TO RELATED APPLICATIONS

This application claims priority to and the benefit of U.S. Provisional Patent Application No. 63/511,331, filed Jun. 30, 2023, which is incorporated herein by reference in its entirety for all purposes.

### STATEMENT REGARDING FEDERALLY SPONSORED RESEARCH OR DEVELOPMENT

This invention was made with Government support under Federal Grant Nos. 5R21GM132709-02 and 1R21GM132709-01 awarded by the Department of Health and Human Services, National Institutes of Health, National Institute of General Medical Sciences. The Federal Government has certain rights to the invention.

### SEQUENCE LISTING

The text of the computer readable sequence listing filed herewith, titled “NWEST_42122_202_SequenceListing.xml”, created Jun. 24, 2024, having a file size of 1,967 bytes, is hereby incorporated by reference in its entirety.

### TECHNICAL FIELD

The present disclosure relates a microfluidic platform for the manipulation of live cells by delivering molecules at various times without killing the cells.

### BACKGROUND

Intracellular delivery of molecular cargoes allows for a wide variety of cell manipulation tasks with applications ranging from cell engineering and gene editing to cell therapy. Given the wide implications of intracellular delivery for fundamental biology research and therapeutics, several technologies have emerged to achieve this task with high efficiency and precision that can be classified into two broad categories: i) direct delivery and ii) carrier-mediated delivery. In direct delivery methods, the cell membrane is permeated by subjecting it to physical disturbances such as mechanical forces, electric fields, or thermal shock. Conversely, carrier-mediated delivery methods rely on chemical interactions between the cell membrane and the nanocarrier, which trigger endocytosis and encapsulation of the molecular cargo. Examples of nanocarriers include lipid-vesicles, spherical nucleic acids, and polymer nanoparticles. Direct delivery methods offer more control and tunability over the membrane permeation process, which can result in tighter dosage precision compared to carrier-mediated methods.

Electroporation is one of the most widely used direct delivery methods because of its ease of use and high throughput. In bulk electroporation systems, cells are placed in a cuvette with embedded electrodes that subject a suspension of cells to an electric field that induces the formation of pores across the cells when the electric potential across the membrane (transmembrane potential (TMP)) reaches a certain threshold. However, high applied voltages are required to achieve threshold TMP in bulk electroporation systems, which result in high cell mortality. Furthermore, the variability in the spatial distribution of the cells in the cuvette result in heterogeneous electric field conditions from cell to cell which reduce dosage precision. To circumvent these limitations, researchers developed electroporation systems that confine the electric field to a small area fraction of the cell membrane by interfacing cells with nanoscale orifices prior to the application of electric pulses. The resulting highly localized electric fields enable the application of much lower voltages compared to bulk systems to achieve threshold TMPs, which results in higher viability. Examples of nanoscale features used in localized electroporation systems include nanochannel membranes, nanopipettes, nanostraws, and nanofountain probes.

Localized electroporation systems have been shown to be efficient in delivering various payloads (e.g., nucleic acids, plasmids, proteins, and nanoparticles) as well as sampling intracellular contents from cells. The performance of localized electroporation systems depends on the electric field conditions, the mechanical properties of the cell membrane, the ionic environment in the surrounding fluid, and the physical properties of the molecular cargo (i.e., size and charge). While these systems offer advantages, they also pose challenges to scalability in number of cells and require high cargo concentrations, which limits their applicability. Consequently, several parameters must be optimized to achieve the desired electroporation performance which include both the efficiency of delivery and the health of the cells.

Current methods for cell manipulation lack the precision and throughput needed for applications ranging from cell therapeutics to disease modeling and drug screening.

U.S. Pat. No. 9,856,448 discloses devices and methods for long-term intracellular access.

U.S. Patent Application Publication No. 2019/0367861 discloses nanostraw well insert devices for improved cell transfection and viability.

### SUMMARY

Efficient and nontoxic delivery of foreign cargo into cells is a critical step in many biological studies and cell engineering workflows with applications in areas like biomanufacturing and cell-based therapeutics. However, effective molecular delivery into cells involves optimizing several experimental parameters. In the case of electroporation-based intracellular delivery there is a need to optimize parameters like pulse voltage, duration, buffer type and cargo concentration for each unique application. Disclosed herein is a protocol for fabricating and utilizing a high-throughput multi-well localized electroporation device (LEPD) assisted by deep learning-based image analysis to enable rapid optimization of experimental parameters for efficient and nontoxic molecular delivery into cells. The LEPD and the optimization workflow presented herein is relevant to both adherent and suspended cell types and different molecular cargo (e.g., DNA, RNA, and proteins). The workflow enables multiplexed combinatorial experiments and can be adapted to cell engineering applications requiring in vitro delivery.

The disclosure provides, in one aspect, an electroporation device comprising a first circuit board including a first plurality of electrodes; a plurality of wells; and a bottomless well plate. Each of the plurality of wells is at least partially received within the bottomless well plate. The electroporation device further includes a second circuit board including a second plurality of electrodes. The bottomless well plate is positioned between the first circuit board and the second circuit board. Each of the first plurality of electrodes is at least partially positioned within one of the plurality of wells.

In some embodiments, the first plurality of electrodes is a plurality of pins.

In some embodiments, the plurality of pins are gold.

In some embodiments, the electroporation device further includes a spacer plate positioned between the bottomless well plate and the first circuit board.

In some embodiments, each of the plurality of wells includes a glass cylinder and a porous membrane.

In some embodiments, the second plurality of electrodes are electrically coupled to the first plurality of electrodes; wherein the electroporation device is configured to apply an electrical pulse to one of the plurality of wells.

The disclosure provides, in one aspect, a system comprising an electroporation device containing a plurality of cells; a voltage generator electrically coupled to the electroporation device; an optical microscope configured to capture an image of the plurality of cells; and a computer including a processor and a non-transitory computer readable memory. The computer analyzes the image based on a machine learning model.

In some embodiments, the machine learning model extracts measurements of the plurality of cells from the image.

In some embodiments, the system further comprises a multimeter electrically coupled to the electroporation device.

In some embodiments, the system further comprises an incubator.

In some embodiments, the electroporation device includes 96 wells.

The disclosure provides, in one aspect, a method comprising culturing cells in an electroporation device; applying a voltage to the electroporation device to deliver a cargo; and analyzing cells post-electroporation.

In some embodiments, analyzing cells post-electroporation includes imaging, scRNAseq, mass spectroscopy, or any combination thereof.

In some embodiments, culturing cells further includes monitoring cells with live cell imaging.

In some embodiments, analyzing cells post-electroporation includes identifying differences in cell physiology and signal with a machine learning model.

In some embodiments, the cargo is nucleic acids, oligonucleotides, plasmid DNA, proteins, protein-spherical-nucleic-acids (pro-SNAs), siRNA, or Cas9ribonucleoprotein complexes (RNP).

Other aspects of the disclosure will become apparent by consideration of the detailed description and accompanying drawings.

### BRIEF DESCRIPTION OF THE DRAWINGS

These features, aspects, and advantages of the present technology will be presented in the following drawings which will aid in a better understanding of the described device. The accompanying figures and examples are provided by way of illustration and not by way of limitation.

FIG. 1A is an exploded view of a multi-well LEPD system design including the following components from top to bottom: PCB board with push-fit electrodes, spacer plate, LEPD devices, bottomless well-plate, and PCB board with electrode pads.

FIG. 1B is a cross-sectional view of the assembled LEPD system of FIG. 1.

FIG. 1C is a photograph of an assembled 24 well LEPD system with LEPD devices inserted in the right-half of the multi-well plate.

FIG. 1D is a photograph of an individual LEPD device placed between a bottom pad electrode and a top stub electrode.

FIG. 1E is a schematic of localized electroporation induced delivery into adherent or suspended cells using LEPD.

FIG. 2A is a schematic of experimental and analysis workflow of a multi-well LEPD system including the following steps (from left to right): i) combinatorial delivery experiments, ii) automated imaging of each well, iii) cell and nuclei identification using deep-learning algorithms, iv) extraction of cellular features (morphology, sub-cellular localization, and dynamics), and v) feature postprocessing and correlation analysis.

FIG. 2B is a schematic of architecture of FCN used to segment the cells and nuclei in an image. An input image is passed through 20 hidden layers consisting of convolution, down-sampling, up-sampling, concatenation, and up-convolution operations that results in a 3-class (e.g., exterior, interior, and border) probability map for each pixel in the image. Layers are color coded based on changes in dimensions (e.g., height and width) down and up the encoder and decoder portions of the network respectively. The thickness of each layer line is proportional to the width of the layer. For a 520×696 grayscale image, there are 7,787,523 trainable parameters in the network.

FIG. 3A illustrates 30 nucleotide long molecules of DNA oligonucleotide (/5ATTO590N/AC TGG TCA CCT GGT CAT CCT GCC GTA ACT G (SEQ ID NO.:1)) tagged with Atto 590 delivered into HeLa using LEPD.

FIG. 3B illustrates 30 nucleotide long molecules of DNA oligonucleotide (/5ATTO590N/AC TGG TCA CCT GGT CAT CCT GCC GTA ACT G (SEQ ID NO.:1)) tagged with Atto 590 delivered into HEK 293T using LEPD.

FIG. 3C illustrates 30 nucleotide long molecules of DNA oligonucleotide (/5ATTO590N/AC TGG TCA CCT GGT CAT CCT GCC GTA ACT G (SEQ ID NO.:1)) tagged with Atto 590 delivered into K562 using LEPD.

FIG. 3D illustrates 30 nucleotide long molecules of DNA oligonucleotide (/5ATTO590N/AC TGG TCA CCT GGT CAT CCT GCC GTA ACT G (SEQ ID NO.:1)) tagged with Atto 590 delivered into Human primary dermal fibroblasts (HDF) using LEPD.

FIG. 3E illustrates 30 nucleotide long molecules of DNA oligonucleotide (/5ATTO590N/AC TGG TCA CCT GGT CAT CCT GCC GTA ACT G (SEQ ID NO.:1)) tagged with Atto 590 delivered into human induced pluri-potent stem cells (hiPSC) using LEPD.

FIG. 3F illustrates 30 nucleotide long molecules of DNA oligonucleotide (/5ATTO590N/AC TGG TCA CCT GGT CAT CCT GCC GTA ACT G (SEQ ID NO.:1)) tagged with Atto 590 delivered into S-HUDEP2 using LEPD.

FIG. 3G is a graph illustrating DNA oligonucleotide delivery efficiency for the investigated cells types of FIGS. 3A-3F.

FIGS. 4A-4D illustrate cell agnostic versatile cargo delivery. Representative images of successful transfection of fluorescent protein encoding plasmids into HeLa (adherent), K562 (cells in suspension), Primary human dermal fibroblasts (HDF) and human induced pluripotent stem cells (hiPSC). hIPSCs were delivered with mCherry encoding plasmid while the other cells were transfected with pmax GFP encoding plasmid. hiPSCs are pseudo colored green. Scale bar=100 μm.

FIG. 4E is a graph of plasmid transfection efficiency of LEPD and Lipofectamine 3000 (LIPO) for various continuous cell lines 24 hours post-delivery. All error bars indicated standard error of the mean of triplicate samples, n_{cell}>100 per sample for all bar plots. *p-value<0.05, ***p-value<0.001.

FIG. 4F is a graph of viability of various continuous cell lines 24 hours post treatment with LEPD and LIPO. Error bars indicated standard error of the mean. All error bars indicated standard error of the mean of triplicate samples, n_{cell}>100 per sample for all bar plots. *p-value<0.05.

FIG. 4G is a graph of plasmid transfection efficiency of LEPD and Bulk electroporation (Bulk EP) for various hard to transfect cell types 24 hours post-delivery. All error bars indicated standard error of the mean of triplicate samples, n_{cell}>100 per sample for all bar plots. **p-value<0.01.

FIG. 4H is a graph of viability of various hard to transfect cell types 24 hours post treatment with LEPD and Bulk electroporation (Bulk EP). All error bars indicated standard error of the mean of triplicate samples, n_{cell}>100 per sample for all bar plots. *p-value<0.05, **p-value<0.01.

FIGS. 5A-5D illustrates delivery of ProSNA and protein activity assay.

FIG. 5A is representative fluorescent micrographs of HeLa cells following 20 s incubation with AF-647-tagged β-gal-SNA (control), electroporation of AF-647-tagged β-gal, or electroporation of β-gal-SNA complex. The DNA of the β-gal-SNA are tagged with FITC. Scale-bars=100 μm.

FIG. 5B is a graph of mean fluorescence intensity (MFI) fold change of AF-647 for different cargo under the same delivery conditions. 3-gal (no DNA attached), SNA1 (0-gal-SNA with 18 DAN strands/β-gal core), SNA2 (0-gal-SNA with 25 DNA strands/β-gal core). Error bars indicate standard error of the mean, N=2 or 3 and *p-value<0.05, n.s.=not significant.

FIG. 5C is representative images of electroporated HeLa cells, with blank samples (control), β-gal, and β-gal-SNA, after X-gal assay. Scale-bars=100 μm.

FIG. 5D is a graph of MFI fold change of X-gal blue precipitate comparing enzyme activity of bare β-gal and β-gal-SNA. Error bars indicate standard error of the mean, N=2 or 3 and *p-value<0.05, n.s.=not significant.

FIG. 6A illustrates CRISPR/Cas9 delivery and gene editing. Representative phase contrast and fluorescent micrographs of K562 cells, 7 days after CRISPR Cas9-gRNA RNP complex and ssODN delivery. The gRNA targeted the endogenous EGFP and the ssODN has a BFP template to facilitate homology directed repair (HDR) (left: Phase contrast, middle: EGFP, right: BFP). Scale bar=100 μm.

FIG. 6B is stacked bar plots of flow cytometry data to show the average efficiency (N=3) of knockout (% of EGFP− cells) and knock-in (% of BFP+ cells). Control refers to untreated cells.

FIGS. 7A-7I illustrate assembly of well-plate electrodes.

FIG. 7A illustrates materials for the assembly procedure including an Au pin and receptacle, top and bottom PCBs, double sided adhesive tape, and a bottomless well-plate.

FIG. 7B illustrates the top PCB used as stencil to mark the locations to cut holes.

FIG. 7C illustrates cutting holes using a biopsy punch.

FIG. 7D illustrates tape after removal of the protective film is aligned with the bottom PCB.

FIG. 7E illustrates a roller is used to compress the tape and the bottom pCB to form a tight seal.

FIG. 7F illustrates the remaining protective film is removed from the adhesive tape.

FIG. 7G illustrates a bottomless well-plate aligned to the bottom PCB pads and pressed firmly to ensure a tight seal.

FIG. 7H illustrates receptacle and pins are inserted into the through-holes of the top PCB.

FIG. 71 is an image of the fully assembled well-plate electrodes.

FIGS. 8A-8L illustrates fabrication of LEPD.

FIG. 8A illustrates materials for the fabrication process of LEPD.

FIG. 8B illustrates double sided tape with 6 mm holes punched through.

FIG. 8C illustrates peeling one side of the tape.

FIG. 8D illustrates placing a porous membrane on a glass slide.

FIG. 8E illustrates pressing porous membrate onto the sticky side of the tape.

FIG. 8F illustrates peeling to expose the other side of the tape.

FIG. 8G-8I illustrates placing cylinders onto the exposed sticky side of the tape and pressing them down.

FIG. 8J-8K illustrates cutting out each LEPD.

FIG. 8L illustrates an individual LEPD after the fabrication process.

FIGS. 9A-9D illustrate representative efficiency results.

FIG. 9A is a representative image of successful transfection of fluorescent protein-encoding plasmids into HeLa (adherent) transfected with a pmax GFP-encoding plasmid. Scale bar represent 100 μm.

FIG. 9B is a representative image of successful transfection of fluorescent protein-encoding plasmids into K562 (cells in suspension) transfected with a pmax GFP-encoding plasmid. Scale bar represent 100 μm.

FIG. 9C illustrates variation of transfection efficiency for HEK 293T cells electroporated in cell culture media (DMEM) and EP buffer using LEPD with respect to pulse voltage. All error bars indicate the standard error of the mean (SEM) of triplicate samples, ncell>100 per sample for all bar plots. All transfection efficiencies are normalized with respect to the highest value of efficiency in each plot. The highest efficiencies for plots in A and B are 71.6, 63.6, respectively.

FIG. 9D illustrates variation of transfection efficiency for HEK 293T cells electroporated in cell culture media (DMEM) and EP buffer using LEPD with respect to pulse duration. All error bars indicate the standard error of the mean (SEM) of triplicate samples, ncell>100 per sample for all bar plots. All transfection efficiencies are normalized with respect to the highest value of efficiency in each plot. The highest efficiencies for plots in A and B are 71.6, 63.6, respectively.

FIGS. 10A-10D illustrate representative viability results.

FIG. 10A is a representative composite fluorescence micrographs of Propidium Iodide (PI) viability assay conducted on HeLa 24 hours after being treated with the LEPD, Pseudo colors: Magenta—Hoechst, Yellow—PI. Scale bar=100 μm.

FIG. 10B is a representative composite fluorescence micrographs of Propidium Iodide (PI) viability assay conducted on HEK 293T 24 hours after being treated with the LEPD, Pseudo colors: Magenta—Hoechst, Yellow—PI. Scale bar=100 μm.

FIG. 10C illustrates viability after plasmid delivery across different voltages: Bar plots showing the mean viability of HEK 293T cells transfected using the LEPD in both DMEM and EP buffer in the voltage range 10 V-40 V. All error bars indicate the standard error of the mean of triplicate samples, n_{cell}>100 per sample for all bar plots.

FIG. 10D illustrates viability of various continuous cell lines 24 hours after treatment with LEPD and LIPO. All error bars indicate the standard error of the mean of triplicate samples, n_{cell}>100 per sample for all bar plots.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in a variety of manners given flexibility of design all of which could not be fully described or illustrated.

### DETAILED DESCRIPTION

All publications, patent applications, patents and other references mentioned herein are incorporated by reference in their entirety. The materials, methods, and examples disclosed herein are illustrative only and not intended to be limiting.

Although any methods and materials similar or equivalent to those described herein can be used in the practice or testing of embodiments described herein, some preferred methods, compositions, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise. The terms “comprise(s),” “include(s),” “having,” “has,” “can,” “contain(s),” and variants thereof, as used herein, are intended to be open-ended transitional phrases, terms, or words that do not preclude the possibility of additional acts or structures.

The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

As used herein, the terms “comprise”, “include”, and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “coupled,” as used herein, is defined as “connected,” although not necessarily directly, and not necessarily mechanically. The term coupled is to be understood to mean physically, magnetically, chemically, fluidly, electrically, or otherwise coupled, connected or linked and does not exclude the presence of intermediate elements between the coupled elements absent specific contrary language.

To expedite the optimization process of electroporation process, a well-plate-based localized electroporation device (LEPD) equipped with multiplexing capabilities is disclosed herein. The multi-well LEPD system allows for tunability in the electric pulse conditions across the rows of the device, and tunability of the molecular cargo or buffer conditions across columns. Furthermore, the LEPD is integrated, in some embodiments, with automated imaging and deep-learning enhanced segmentation to analyze the morphology of cells following delivery in addition to delivery efficiency and viability. The integrated approach provides a flexible platform for cell manipulation and analysis.

The present disclosure provides a system capable of multiplexed delivered of any biomolecule without eliciting cellular responses or downstream changes in cell state affording the capability of intracellular delivery at multiple time-points. The system includes well arrays, electronics for application of a variety of electric pulses to open pores temporarily in the cell membrane. Since the cell membrane heals after the application of the electric pulses, the process is non-destructive and minimally affects cell functioning. In some embodiments, the platform is integrated with an inverted optical microscope within an incubator allowing for multipoint, multiplexed and combinatorial perturbation of millions of cells with controlled introduction of external molecular cargo. This enables applications such as cellular engineering for cell therapies, disease modeling and drug screening.

The present disclosure provides a microfluidic platform (the Localized Electroporation Device or LEPD) that can culture adherent and suspended cells in 24-well plates. Deliver exogenous molecules (e.g., plasmids, mRNA, proteins, and peptides) into cells in each well is done efficiently, uniformity, and with high viability. The device facilitates long-term onchip culture and monitoring (e.g., through live cell imaging and AI algorithms) cell populations over days. The multi-well plate format enables the processing of millions of cells with multiplexed delivery conditions simultaneously. The high-throughput and combinatorial capabilities of this design significantly reduce the time required to optimize the experimental conditions needed to achieve efficient molecular delivery into different cell types. Furthermore, in some embodiments, the multi-well LEPD platform is integrated with deep learning enhanced imaging to track the cells and extract hundreds of measurements with single-cell resolution.

The parallelized localized electroporation platform disclosed herein with multiplexing capabilities and AI-enhanced image analysis allows for the rapid optimization of experimental conditions required for efficient intracellular delivery and for robust comparisons of different cell treatments. Furthermore, the throughputs achieved with this device exceed previous demonstrations of localized electroporation platforms, which is essential for applications that require the rapid processing of large numbers of cells such as gene-editing and T-cell immunotherapy. The leveraging of AI to analyze image data significantly improves both the accuracy and turnaround time of the process as it enables quick identification and quantification of even subtle differences in characteristics of cell physiology and signal between cells exposed to different experimental conditions. Also, the LEPD disclosed herein introduces much less stress in cells than bulk electroporation, as demonstrated using single-cell RNA-sequencing. Furthermore, cells in the LEPD can be monitored over days by leveraging AI image analysis, which significantly improves both the accuracy and turnaround time of the process as it enables quick identification and quantification of even subtle differences in characteristics of cell physiology and signal between cells exposed to different experimental conditions.

Using the multi-well LEPD, the optimization is demonstrated of plasmid delivery into a range of immortalized cell lines as well as into cells that are hard to transfect (primary/stem cells) with high transfection efficiencies (40-85%) while maintaining high viability (82-95%). Depending on the cell type, the LEPD is either superior or comparable to commercially available delivery systems in terms of performance metrics like delivery efficiency, viability, precision, and control over dosage.

As disclosed herein, the multi-well LEPD is employed to achieve functional gene knockdown in a clinically relevant cell type, human immune pluripotent stem cells (hiPSCs), and used the deep learning framework to analyze changes in morphological features and protein expression in the genetically perturbed cells with single cell resolution. It shows the potential of the LEPD system to be used in studies related to stem cell engineering or reprogramming wherein outcomes like expression levels of multiple proteins, sub-cellular localization and morphological changes are important characteristics to be closely monitored.

Multi-Well Plate LEPD (Localized-Electroporation Device): The design of the multiplexed localized-electroporation device (LEPD) allows for the simultaneous application of electrical pulses across independently addressable wells in a multiwell plate format, which enables the rapid optimization of electroporation protocols. The multiplexed LEPD can be used to deliver a diverse range of molecular cargoes (proteins, mRNA, plasmid DNA etc.) directly into the cytosol of different cell types, including adherent and suspended cells, with a high degree of control while preserving viability.

In one embodiment, the LEPD architecture consists of individual cell-culture wells which are composed of glass cylinders (ID: 6 mm, OD: 8 mm) bonded to polycarbonate (PC) track-etched membranes. Each culture well can hold up to approximately 50,000 cells, but this number can vary depending on the diameter of the well, size of the cells, and the plating density. The culture wells are placed in a well-plate assembly that consists of a conductive substrate (ITO-coated glass or PCB with gold pads) at the bottom, attached to a bottom-less well-plate, a plastic transfer layer for facile transfer of the culture wells, and a PCB with a pattern of plated through-holes, concentric to the wells, where individual electrodes (pin or nail-head stub) are inserted using push-fit receptacles.

For intracellular delivery experiments, a small volume (e.g., approximately 2-4 uL) of the delivery cargo of interest is dispensed on the bottom conductive substrate, the media in the wells is replaced with electroporation buffer, transferred to the well-plate electrode assembly using the transfer piece, and covered by the top PCB layer which allows for the precise immersion of the electrodes are in the culture-wells. The multi-well-electrode assembly is connected to an electronics box equipped with a function generator that can be programmed to generate pulse trains of a variety of pulse shapes (square, bi-level, exponential), voltage amplitudes (0-60 V), frequencies (max 400 Hz), pulse durations (0.5-10 ms), and number of pulses. Individual wells can be programmed independently to allow for the combinatorial application of different pulse conditions. Cells are permeabilized during the application of electric pulses if the transmembrane voltage (TMV) exceeds a threshold (e.g., approximately 0.2 V) that results in the formation of hydrophilic pores which is energetically favorable. The electric field is confined to a nanoscale region of the cell membrane via the nanochannels of the PC membrane that results in pore formation in a small area fraction (approximately 0.06-15%) of the cell membrane while requiring low applied voltage amplitudes (approximately 10 V) relative to bulk electroporation systems (approximately 500-1000 V). After the localized-electroporation delivery experiments are conducted, the culture wells can be transferred to a transparent well-plate for imaging or other downstream assays.

The modular multi-well LEPD design allows for facile integration with robotic fluid handlers and imaging systems, which can be leveraged to scale-up cell engineering processes. The automation of imaging with the well-plate format allows for integration with deep-learning-based computer vision software to segment cells, their intracellular compartments, and delivered fluorescent cargoes for the downstream analysis of shape, intensity, and texture features. The ability to extract various features from the images enables a more comprehensive analysis of the cell phenotype following electroporation in addition to the standard metrics of delivery efficiency and cell viability.

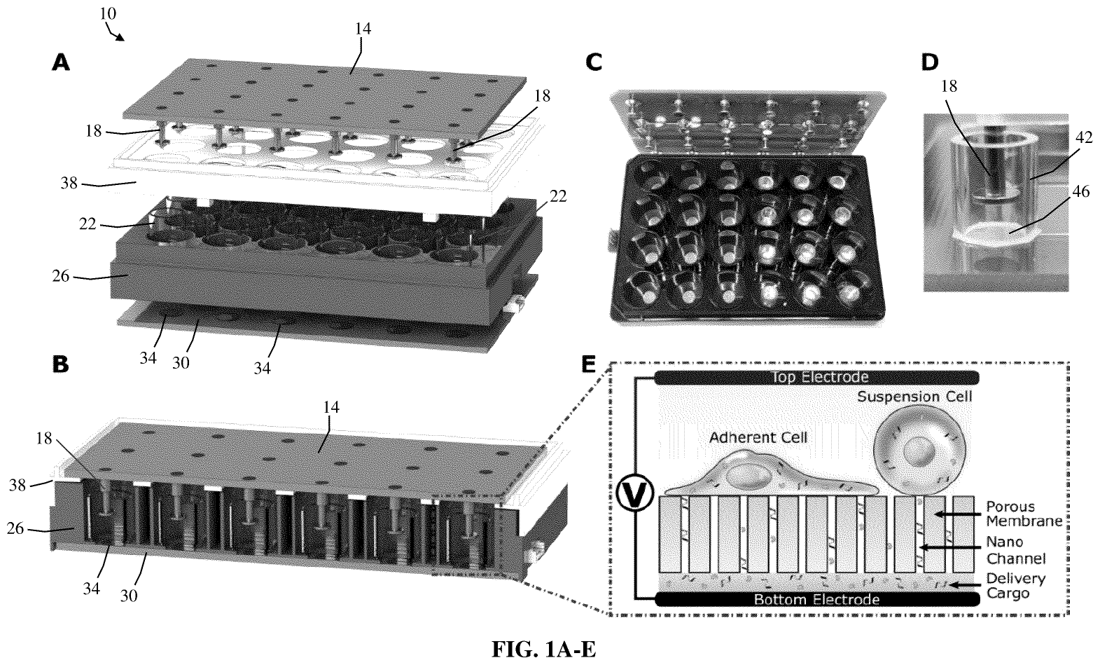

With reference to FIG. 1A, the electroporation device 10 illustrated includes a first circuit board 14 including a first plurality of electrodes 18, a plurality of wells 22, and a bottomless well plate 26. In the illustrated embodiment, each of the plurality of wells 22 is at least partially received within the bottomless well plate 26. The electroporation device 10 further includes a second circuit board 30 with a second plurality of electrodes 34. The bottomless well plate 26 is positioned between the first circuit board 14 and the second circuit board 30. In the illustrated embodiment, the electroporation device 10 further includes a spacer plate 38 positioned between the bottomless well plate 26 and the first circuit board 14.

With reference to FIG. 1B, each of the first plurality of electrodes 18 is at least partially positioned within one of the plurality of wells 22. In the illustrated embodiment, the first plurality of electrodes 18 is a plurality of pins. In some embodiments, the plurality of pins are made of gold. Each of the second plurality of electrodes 34 are is aligned and electrically coupled to one the first plurality of electrodes 18. As such, the electroporation device 10 is configured to apply an electrical pulse individually to any one of the plurality of wells 22.

With reference to FIG. 1D, each of the plurality of wells 22 includes a glass cylinder 42 and a porous membrane 46.

Device Fabrication: The fabrication of the LEPD, in some embodiments, is broken down into the assembly of three modules: (i) well-plate with electrode (PCB or ITO) substrate), (ii) top PCB with protruding electrodes, and (iii) cell-culture wells.

In one embodiment, materials used to assemble the bottom well-plate-electrode sub-assembly include: polystyrene well-plate with through-holes (24 or 96 well-plate), a customized PCB board (80×120 mm) containing an array of gold pads that matches the well-plate geometry (e.g., 24 well: 4 rows, 6 columns, 6 mm diameter, 19.5 mm spacing), a right-angle header pin housing, and a double-sided pressure adhesive tape (Adhesives Research). In one embodiment, steps for assembling the bottom well-plate-electrode sub assembly include: STEP 1: Preparation of adhesive tape, cut tape to the dimensions of the bottom PCB (80×120 mm) and perforate holes to match the pattern of the well-plate (e.g., 24 well: 4 rows, 6 columns, 15 mm diameter, 19.5 mm spacing); STEP 2: Assembly of components: (a) remove one side of the adhesive tape protective laminate cover and attach the tape to the top surface of the PCB, (b) remove the laminate cover from the remaining side of adhesive tape and attach the through-hole well-plate on top, (c) apply pressure to form a robust seal; and STEP 3: Pin housing assembly, solder the right-angle header pin housing to the plated through holes on the PCB.

In one embodiment, materials used to assemble the top well-plate-electrode sub-assembly include: a customized PCB board (80×120 mm) containing an array of plated-through-holes with that matches the well-plate geometry (e.g., 24 well: 4 rows, 6 columns, 3 mm diameter, 19.5 mm spacing), press-fit receptacles (Mill-Max), Au-coated pins (2.0 mm diameter, 19.0 mm length), and a right-angle header pin housing. In one embodiment, steps for assembling the top well-plate electrode sub-assembly includes: STEP 1: Electrode assembly, (a) place press-fit receptacles into the plated-through-holes of the PCB and insert with force, (b) insert the pins into the receptacles; STEP 2: Pin housing assembly, solder the right-angle header pin housing to the plated through holes on the PCB.

In one embodiment, materials used to assemble the nanochannel-culture-wells: glass cylinders (6 mm ID, 8 mm OD), track-etched PC membrane (it4ip) with nanochannels (e.g., 400 nm diameter, 2×106 pores/cm2), and a double-sided pressure adhesive tape (Adhesives Research). In one embodiment, steps for assembling the nanochannel-culture wells includes: STEP 1: Cleaning the glass cylinders: (a) immerse the glass cloning cylinders in acetone and sonicate for 10 minutes, (b) rinse with ethanol and deionized distilled water (DDW) and (c) dry with N2 gas; STEP 2: Preparation of adhesive tape: perforate holes (˜10 holes for a 75 mm×25 mm area) through the double-sided adhesive tape; STEP 3: PC membrane preparation: place the track-etched PC membrane on a clean glass slide and place in oxygen plasma cleaner for 5 minutes; STEP 4: Assembly: (a) remove one side of the protective film from the adhesive tape, (b) place the PC membrane on top ensuring it remains flat by using the rigid microscope slide as support, (c) invert the PC membrane-adhesive tape and remove the remaining protective laminate film from the adhesive tape, (d) place the glass cylinders on the exposed adhesive tape aligned at the location of the pre-cut holes, (e) apply pressure on the top of the glass cylinder to form a robust seal, and (f) cut the tape to separate each cylinder.

Experiments using the LEPD: The LEPD is a versatile tool that allows for the nondestructive poration of adherent and suspended cells for intracellular delivery or sampling of cytosolic contents for analysis. The experimental workflow, in one embodiment, includes three main steps: i) cell culture, ii) electroporation-induced delivery or sampling, and iii) post-electroporation cell analysis (e.g., imaging, scRNAseq, or mass spectroscopy). Because of the modular design and compatibility with imaging, the LEPD system can be used for a wide range of user-customized cell engineering workflows.

On-chip cell culture: The nanoporous polymer membranes of the LEPDs are first coated with appropriate extracellular matrix protein (e.g., fibronectin) for cell lines and fibroblasts at a concentration of 1-5 μg/cm2; vitronectin for hiPSCs at a concentration of 0.1-1 μg/cm2) and incubated for 1 h. For The devices are left uncoated for cells in suspension. Following this the devices are washed with PBS twice. For adherent cell types, 5,000 to 30,000 cells of interest are dispensed into the LEPD wells in 100 μL of the corresponding culture medium. The cells are then cultured on the PC-membrane surface overnight in the incubator (at 37° C. with 5% CO2) to promote cell adhesion and tight nanopore-cell membrane contact before electroporation the next day. For suspension cells, 15,000-60,000 cells are introduced in the LEPDs containing electroporation buffer and centrifuged at 150×g for 5 min to establish tight cell contact with the nanopores before electroporation. Post electroporation, the LEPD arrays are transferred to 24 well plates with the appropriate medium depending on the cell type, in an incubator and cultured for downstream imaging or assays.

Electroporation: The media in the LEPDs is replaced with electroporation buffer (iso-osmolar or hypo-osmolar) prior to electroporation experiments. A 2-5 μL droplet of solution containing the cargo at the desired concentration is dispensed at the center of the gold pads of the bottom PCB layer of the well plate LEPD system. Then, individual LEPDs containing the cells in the electroporation buffer are placed on the droplets containing the cargo. The droplets form a thin film between the bottom electrode and the nanoporous polymer membrane of the LEPDs containing the cells. Finally, the top PCB layer containing the gold coated electrodes (pins/stubs) is placed over the LEPDs forming a closed electrical circuit as the top electrodes are submerged in the electroporation buffer inside the culture wells. To ensure proper electrical connection prior to electroporation, a multimeter integrated into the electronics box (Infinitesimal LLC) is used to measure resistance. A function generator (Infinitesimal LLC) is programmed to apply the electroporation pulses of various shapes (e.g., square, bi-level, exponential) and customizable pulse parameters (voltage amplitude, pulse-time, frequency, and pulse number). Different pulse parameters can be applied to different regions of the LEPD depending on the design of the top PCB electrode. For multiplexing and optimization, a top PCB with individually addressable rows or wells is used. For high-throughput applications following optimization, a top PCB with a common electrode is used to apply the same pulse type across the well-plate. After electroporation is complete, the individual LEPD wells containing the cells can be transferred to a separate standard well-plate to exchange the electroporation buffer and for downstream processing.

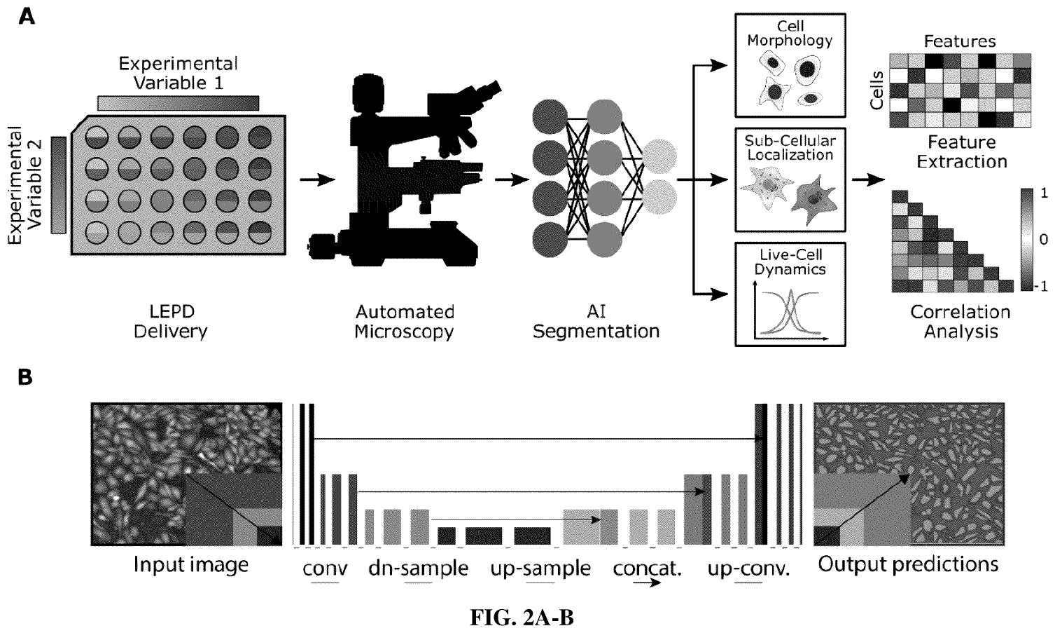

Automated Image Analysis Workflow: To extract morphology and intensity information from individual cells in each LEPD following the multi-well delivery experiments, we developed an automated imaging and analysis workflow. The workflow (FIG. 2A) involves imaging each well using a microscope equipped with a motorized stage, segmenting the cells in each image using an AI-detection pipeline, and extracting shape and intensity features from each cell for analysis using statistical tools. A fully convolutional network (FCN) with a U-Net architecture can be trained to segment objects (cells, intracellular components, fluorescent probes) in fluorescent or phase contrast micrographs. In one embodiment, the U-Net contains 20 hidden layers (FIG. 2B) arranged in an encoder-decoder scheme that outputs a multiclass probability used to classify each pixel in the image into three classes (interior, boundary, or exterior). Once the cell or nuclei objects are identified in each image, they are passed through a feature extraction pipeline (CellProfiler) that can measure various intensity, shape, texture, and environment features. Each feature is transformed using the generalized log (g log) method and normalized using robust Z-score standardization to enable comparison of features utilizing the same scale while preventing outliers from skewing the standardization. To reduce the number of features and improve the interpretability of the data, feature reduction methods like PCA or correlation matrices may be used. Furthermore, a dimension-reduction method, uniform manifold approximation and projection (UMAP) or t-distribution stochastic network embedding (t-SNE), can be used to visualize the high-dimensional data on a corresponding 2D projection. This workflow is utilized to calculate performance metrics, such-as delivery/transfection efficiency, and viability, in addition to other metrics related to cell morphology (e.g., area, circularity, and eccentricity) and analyze their relationships. As a result, the automated image analysis pipeline enabled the correlation of experimental inputs to the phenotypic outputs across devices in the multi-well LEPD.

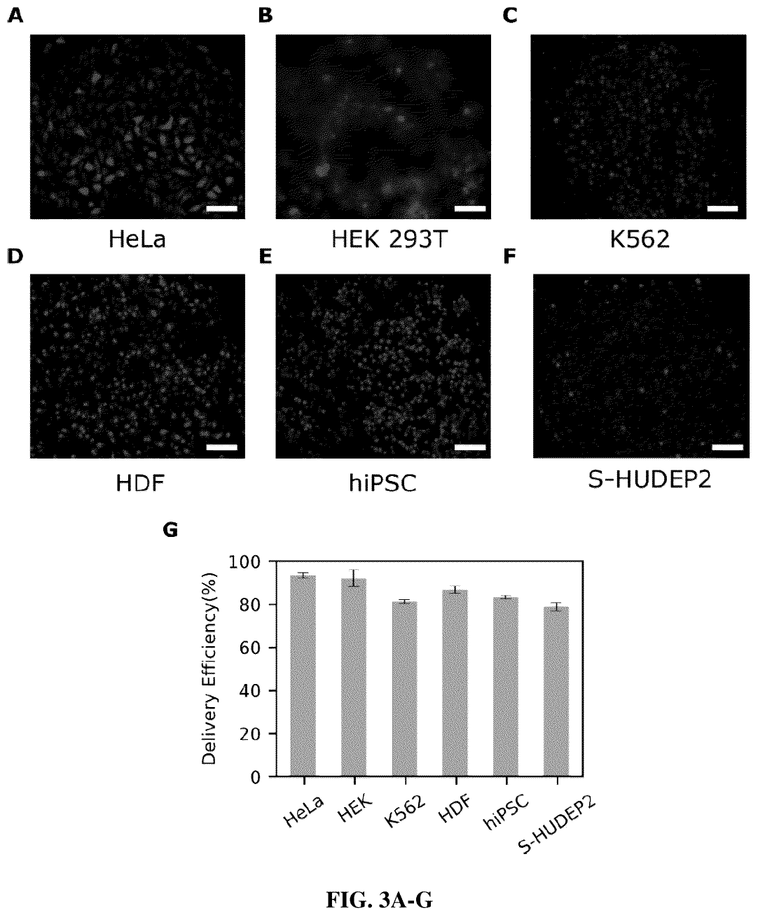

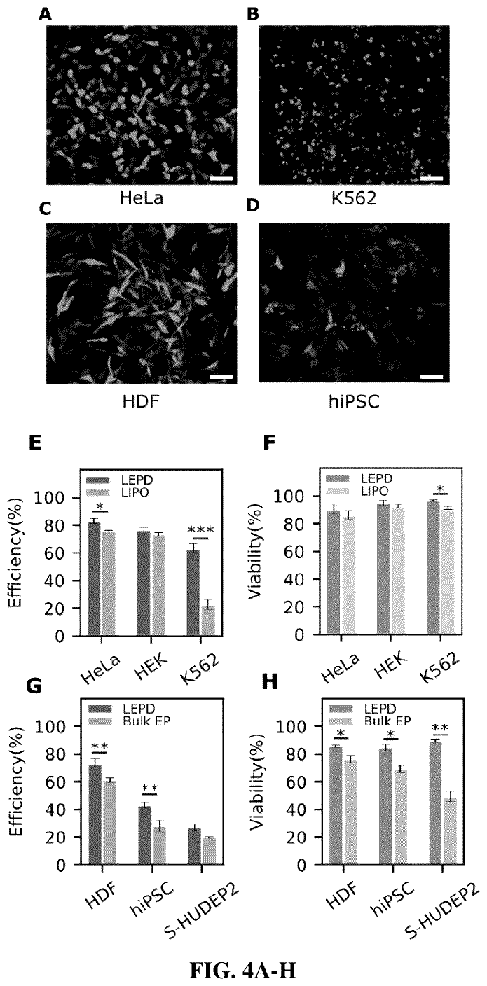

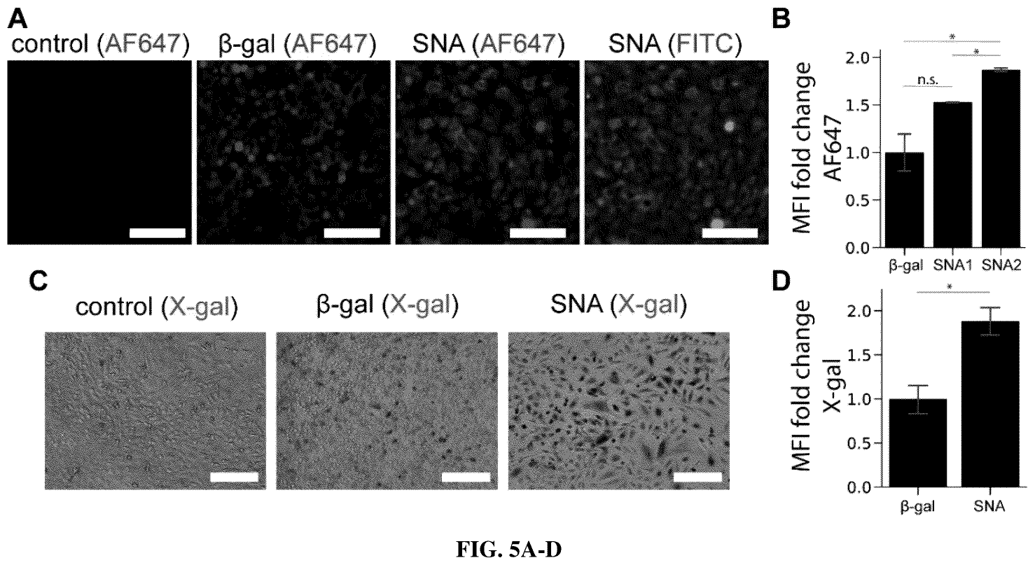

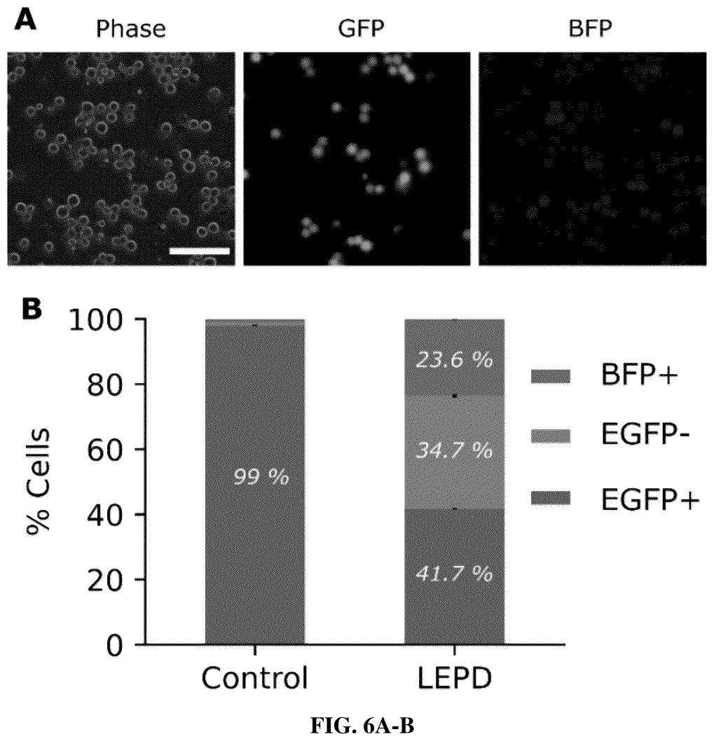

Nondestructive Delivery of Molecular Cargoes: The LEPD system can be used to optimize the delivery of different molecular cargoes into different types of cells. Optimization is required for each delivery cargo and cell type to achieve high performance (e.g., delivery efficiency and viability) due to the large variability in size and charge of the different cargoes as well as differences among cell types in terms of sensitivity to perturbation, size, and cell-membrane tension. The multi-well LEPD has been used to deliver the following molecules with high efficiency: nucleic acids, oligonucleotides (FIG. 3), plasmid DNA (FIG. 4), proteins (FIG. 5), protein-spherical-nucleic-acids (pro-SNAs) (FIG. 5), siRNA, and Cas9ribonucleoprotein complexes (RNP) (FIG. 6).

Regarding Applications: Using the multi-well LEPD disclosed herein, optimization of plasmid delivery into a range of immortalized cell lines is demonstrated as well as into cells that are hard to transfect (primary/stem cells) with high transfection efficiencies (40-85%) while maintaining high viability (82-95%). Depending on the cell type, the LEPD is either superior or comparable to commercially available delivery systems in terms of performance metrics like delivery efficiency, viability, precision, and control over dosage. Using the multi-well LEPD disclosed herein, functional gene knockdown is achieved in a clinically relevant cell type, human immune pluripotent stem cells (hiPSCs), and the deep learning framework is used to analyze changes in morphological features and protein expression in the genetically perturbed cells with single cell resolution. It shows the potential of the LEPD system to be used in studies related to stem cell engineering or reprogramming wherein outcomes like expression levels of multiple proteins, sub-cellular localization and morphological changes are important characteristics to be closely monitored. Direct delivery of protein-core spherical nucleic acids is demonstrated herein used in intracellular live cell biosensing and biological development (e.g., immunotherapy and enzyme replacement therapy). Also, delivery of siRNA is demonstrated to knock down proteins and affect cell function. Applications include cell therapies, disease modeling, drug screening.

The advantages of the present disclosure include: (i) a high-throughput optimization of cell transfection; (ii) accurate delivery of payload including ratiometric delivered of biomolecules with much more precision than bulk electroporation or lipofection; (iii) the microfluidic environment created using micro-wells provides a means for long term cell culture, gene editing, monitoring, and analysis; and (iv) provides a platform for simultaneous perturbation of cells at multiple timepoints. Current methods do not combine perturbation and AI image analysis on the same platform.

The present disclosure provides methods for efficient manipulation of large cell populations with precision of delivered payload and minimum cell state perturbation. The platform allows for non-destructive high throughput and combinatorial experiments. This enables efficient determination of optimal transfection conditions as a function of cell type and molecular cargo. As such, the system disclosed herein can be integrated in cell therapy workflows. The new platform disclosed herein provides for the manipulation of cells with high throughput and efficiency would enable discovery of cell therapies, identification of novel genetic targets and drugs.

### Example Detailed Protocol

### Materials and Reagents:

### 1. Well-Plate Electrode Assembly

a. Custom printed circuit board (PCB) with gold electrode pads
b. Custom PCB with through-holes
c. Push-fit PCB receptacle (Mill-Max, catalog number: 0350-O-15-15-07-27-10-0)
d. Au-coated pins (straight pin or nail-head pin) (Mill-Max, catalog number: 3580-1-00-15-00-00-03-0)
e. Bottomless well-plate (Greiner Bio-One, catalog number: 662000-06)
f. Biopsy Punch, 12 mm (Acuderm, catalog number: P1250)
g. Double-sided pressure adhesive tape (Adhesives Research, catalog number: MH-90880)
h. Razor blade
i. Marker

### 2. Device Assembly

a. Track-etched polycarbonate membranes (Itpi4, catalog number: 1000M25/620N401/7525)
b. Cloning cylinders (MilliPore Sigma, catalog number: CLS31668)
c. Double-sided pressure adhesive tape (Adhesives Research, catalog number: MH-90880)
d. Biopsy Punch, 6 mm (Miltex, catalog number: 33-36)
e. Scissors
f. 70% EtOH/H20

### 3. Electroporation

a. Low conductivity electroporation buffer (Eppendorf, catalog number: EW-36205-62)

### 4. Surface Treatment and Cell Culture

a. Fibronectin from Human Plasma (0.1% Solution, Sigma Aldrich, catalog number: F0895)
b. Dulbecco's Modified Eagle Medium (DMEM), High Glucose, Pyruvate (Gibco, catalog number: 11995065)
c. Fetal Bovine Serum (FBS), (Gibco, catalog number, A3160501)
d. Penicillin-Streptomycin (Pen-Strep) (10,000 U/mL) (Gibco, catalog number: 15140148)
e. Trypsin—EDTA (0.25%), Phenol Red (Gibco, catalog number, 25200056)
f. Nunc™ Cell-Culture Treated Multidishes, 6 well plates (Thermo Fisher Scientific, catalog number: 140675)
g. Phosphate Buffered Saline (PBS) (Gibco, catalog number: 10010023)
h. HeLa cells (ATCC, catalog number: CCL-2)

### 5. Plasmid

a. eGFP plasmids (stock concentration 1000 ng/μl)

### 6. Automated Imaging

a. Hoechst 33342 nuclear stain (Invitrogen, catalog number: H3570)

### Equipment

1. Electronics box for pulse generation and resistance measurements along with pulse control software (Infinitesimal LLC)
2. Inverted microscope (Nikon Ti-E) equipped with 4×, 10×, and 20× objectives, fluorescent light source, epi-filter cubes, X-Y-Z motorized stage, and a CMOS camera (Andor Zyla) for image acquisition
3. Computer for setting of pulse parameters and image processing
4. Laboratory centrifuge (Thermo Fisher Scientific, Catalog number: 75007200)

### Example Procedures

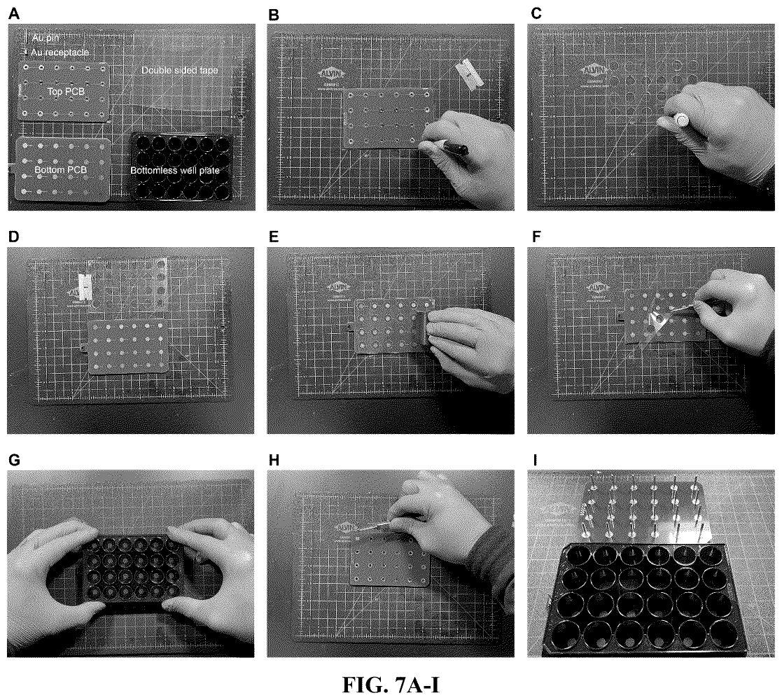

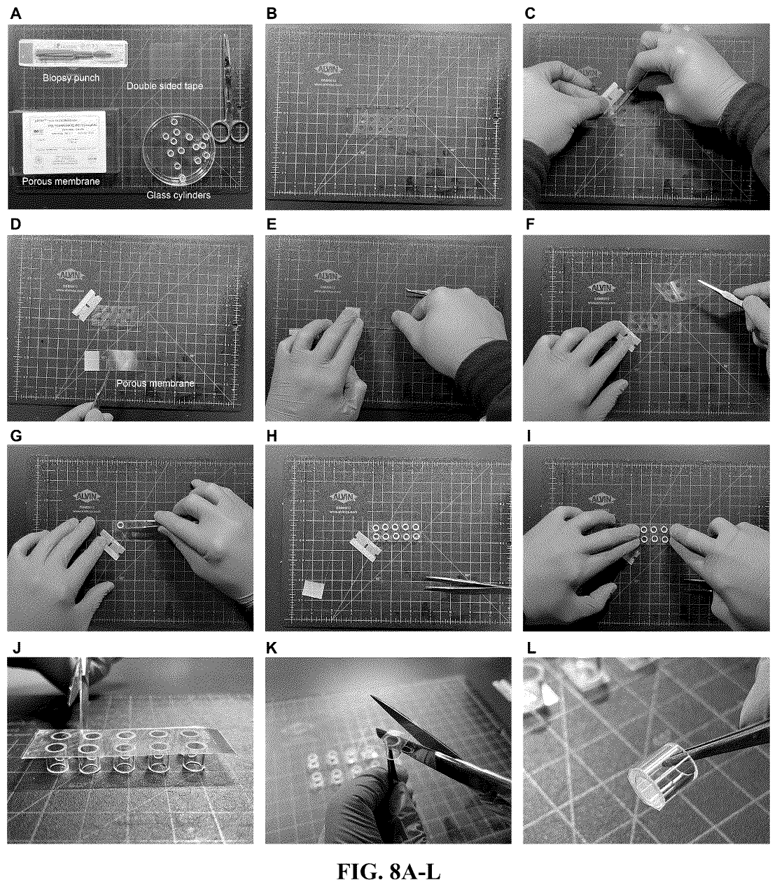

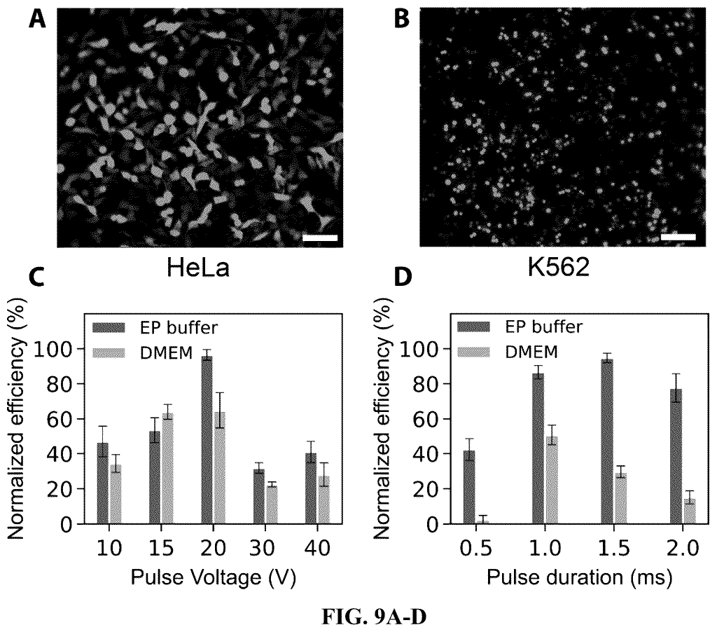

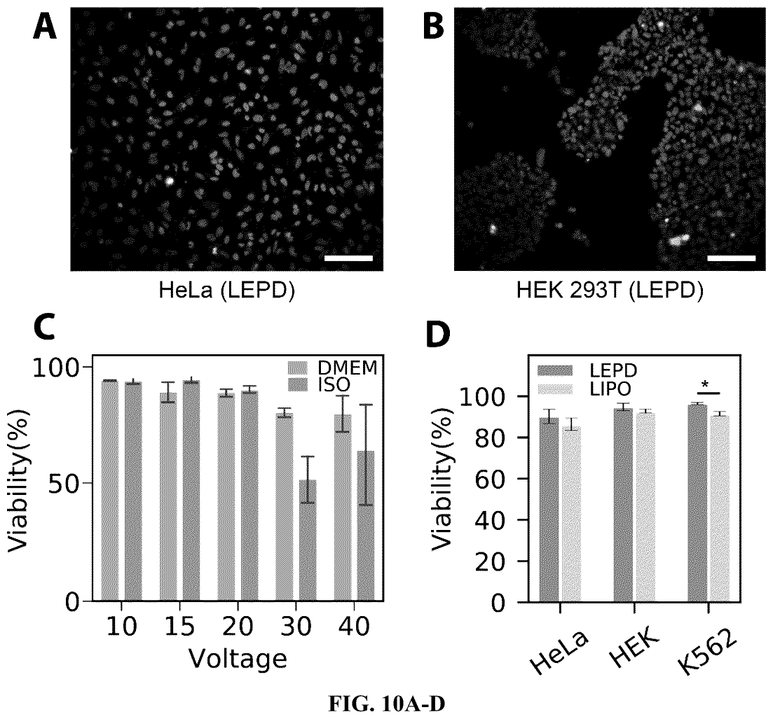

Well-plate electrode assembly: With reference to FIGS. 7A-7I, an example well-plate electrode assembly process is illustrated and includes the following:
STEP 1. Cut a piece of double-sided adhesive tape to match the dimensions of the bottom PCB by holding down the PCB on the double-sided adhesive and using a razor blade to cut at the edges of the PCB.
STEP 2. Place the top PCB on top of the tape and use it as a stencil to mark the location of the wells using a marker (FIG. 7B).
STEP 3. Use a plotter cutter or a biopsy punch (10-15 mm) to cut holes at the markings from the previous step (FIG. 7C).
STEP 4. Remove one side of the adhesive tape by carefully inserting a razor blade at a corner and pulling the film with clean tweezers. Place the bottom PCB adjacent to the tape facing up (FIG. 7D).
STEP 5. Use two tweezers to hold the tape from both sides and carefully align the tape to the bottom PCB by ensuring the holes on the tape are concentric to the Au pads on the PCB and place the tape on the surface of the PCB.
STEP 6. Press down firmly on the adhesive tape to ensure a tight seal by using a rigid cylinder to roll over the adhesive film while applying force onto the surface (FIG. 7E).
STEP 7. Remove the remaining protective film from the double-sided adhesive tape by separating the film from the adhesive using a razor blade and removing the laminate with a tweezer (FIG. 7F).
STEP 8. Place the bottomless well-plate on the surface while ensuring proper alignment of the wells to the Au pads. Firmly press down on the well-plate to ensure a tight seal (FIG. 7G).
STEP 9. Insert the receptacles onto the holes of the top PCB firmly and insert the Au-coated pins into the receptacles (FIG. 7H and FIG. 7I).
STEP 10. Position the pin headers at designated connection points on the top PCT and solder PCB pin headers to the top PCB to connect assembled well-plate electrodes to the function generator.

Well device assembly: With reference to FIGS. 8A-8L, an example device assembly process is illustrated and includes the following:
STEP 1. Immerse the glass cloning cylinders in acetone and sonicate for 10 minutes. Rinse the cylinders with ethanol and deionized distilled water (DDW) and dry with N2 gas.
STEP 2. Use a 6 mm biopsy punch to cut holes (˜10 holes for a 75 mm×25 mm area) through the double-sided adhesive tape (FIG. 8B).
STEP 3. Remove one side of the protective film from the adhesive tape by using a razor blade to separate the laminate from the tape at a corner and carefully removing the laminate with tweezers (FIG. 8C).
STEP 4. Place the track-etched PC membrane on a clean glass slide and place in oxygen plasma cleaner for 5 minutes.
STEP 5. Gently place the PC membrane on top of the tape from step 3 ensuring it remains flat by using the rigid microscope slide as support (FIG. 8D and FIG. 8E).
STEP 6. Flip the PC membrane-adhesive tape and remove the remaining protective film from the adhesive tape using a razor blade and a tweezer (FIG. 8F).
STEP 7. Place the cloning cylinder on the exposed adhesive tape aligned at the location of the pre-cut holes (FIG. 8G and FIG. 8H).
STEP 8. Press down on the top of the glass cylinder to activate the pressure-adhesive and ensure a good seal between the assembled layers (FIG. 8I). Repeat this step for each hole cut in the adhesive tape.
STEP 9. Use scissors to cut the tape to separate each cylinder (FIG. 8J), and subsequently trim the edge of the tape that surrounds each cylinder (FIG. 8K and FIG. 8L). Spray the scissors with 70% ethanol in DI water to prevent tape from sticking on the surface of the scissors.
STEP 10. Place the devices in a well-plate dish exposed to UV light for 4 h.

Surface treatment and cell culture: an example process is illustrated and includes the following:
STEP 1. Prepare fibronectin solution for surface treatment by diluting the stock solution in PBS (20 μL of fibronectin 157 in 1 mL of PBS) to obtain a final concentration of 20 μg/mL.
STEP 2. Add 100 μL of the prepared solution to each LEPD and incubate for 1 h at room temperature inside a biosafety cabinet.
STEP 3. Carefully discard the excess, unbound fibronectin solution from each LEPD and wash them with 100 μL PBS 2 times. This ensures that only the adhered fibronectin layer is retained on the devices, which are now ready for cell culture.
STEP 4. Prepare complete cell culture media by adding 10% (50 mL) FBS and 1% (1 mL) Pen-Strep to 449 mL DMEM.
STEP 5. Use this media for HeLa cell culture in 6 well plates.
STEP 6. Seed cells at a density of 3×105 cells/well and use 2 mL media per well for culturing.
STEP 7. Wait till cells reach confluency (1×106 cells/well) in the well plates before dissociating them for plating in the LEPDs.
STEP 8. When cells are well adhered and confluent, gently discard the cell culture media in the 6 well plates and add 1 mL of warm (37° C.) trypsin-EDTA to each well.
STEP 9. Incubate (at 37° C. with 5% CO2) for 5 mins.
STEP 10. Gently pipette the trypsin-EDTA to detach and dissociate the cells and transfer to cell suspension to a 15 mL falcon tube.
STEP 11. Add 4 mL of the complete cell culture media to the cell suspension to neutralize the trypsin.
STEP 12. Centrifuge the cells at 300×g for 5 mins.
STEP 13. Discard the supernatant and add fresh 1 ml complete cell culture media and resuspend the cells.
STEP 14. Count the cells using a hemocytometer.
STEP 15. Dilute the cells if necessary to obtain a final cell suspension concentration of 200 cells/μL.
STEP 16. Add 20,000 HeLa cells in each LEPD by pipetting 100 μl of the cell suspension solution.
STEP 17. Pipette 100 μL of additional complete cell culture media into each well.
SETP 18. Culture the cells on the membrane surface overnight in an incubator (at 37° C. with 5% CO2) to promote cell adhesion and tight cell membrane and nanopore contact.
STEP 19. Electroporate the adhered cells on the LEPDs the next day.
STEP 20. Note that this process can be adapted for various adherent and suspension cell types. The surface treatment, cell seeding, and culture conditions must be optimized accordingly.

Delivery into Suspension Cells: an example process is illustrated and includes the following:
STEP 1. Check the cells in the microscope to ensure the cells look viable and have proper morphology.
STEP 2. Count the cell density using a hemocytometer or an automated cell-counter.
STEP 3. Take the appropriate volume of cells from the media to plate between 30,000-50,000 cells per device (e.g., 24 devices require ˜1.2 million cells).
STEP 4. Take the appropriate volume of media that contains the desired number of cells calculated from the cell density obtained from step 2 and place the cells in a Falcon tube.
STEP 5. Place the tube containing the cells in a centrifuge and add a counterweight to balance. Centrifuge for 5 min at 150×g.
STEP 6. Gently remove the cell media from the Falcon tube, leaving the pellet of cells. Add electroporation buffer to the tube: 100 μl of electroporation buffer per device (2.4 mL for 24 devices). Mix the cells in the electroporation buffer.
STEP 7. Dispense 100 μl of electroporation buffer containing the cells in each device inside of a well-plate.
STEP 8. Place the well-plate which contains the devices with cells into the centrifuge. Place a counterweight (well-plate with fluid in each well [˜200 μl per well]) in the opposite chamber of the centrifuge and centrifuge at 150 g for 5 min.

Optimization of plasmid delivery: an example process is illustrated and includes the following:
STEP 1. Prepare eGFP plasmid solutions of concentration ranging from 100 ng/μL to 350 ng/μL with a step size of 50 ng/μL. The minimum volume of each solution should be 5 μL.
STEP 2. Take the electroporation buffer out of 4° C. storage and allow it to come to room temperature.
STEP 3. Transfer the LEPDs having the cells from the incubator into a bio-hood, carefully pipette out all the media from the LEPDs and add 200 μL of the electroporation buffer to each LEPD.
STEP 4. If using non-adherent cells, place the LEPDs from step 3 in a well plate and centrifuge them at 150×g for 5 min.
STEP 5. Using a 10 μL pipette, place a 5 μL droplet of the 100 ng/μL eGFP plasmid solution on the bottom gold electrodes of the wells of column 1 of each row of the 24-well LEPD system. Repeat this for the higher concentration eGFP plasmid solutions for the remaining columns going up in concentration to 350 ng/μL for the 6th column.
STEP 6. Using a pair of sterile tweezers gently place the LEPDs having the cultured cells onto the wells having the droplet of delivery plasmid on the bottom gold electrode with one LEPD device per well.
STEP 7. Set the pulse conditions on the electronics software (e.g., provided by Infinitesimal LLC) to a bi-level pulse with pulse parameters: V1 ranging from 10 to 40 V; V2 fixed at 10 V; T1 at 0.5 ms; T2 ranging from 0.5 to 2.5 ms; frequency set at 20 Hz; and pulse count between 100 and 800.
STEP 8. Connect the positive terminal of the electronics box to row 1 of top plate, and the negative terminal to the bottom plate using cables with alligator clip connectors.
STEP 9. Check the resistance of the system using the software (Infinitesimal LLC) and check for loose connections if the resistance is not in the 1-2 MΩ range and is unstable.
STEP 10. Use the electronics software to apply the pulse, the cells in the devices in row 1 will be subjected to localized electroporation.
STEP 11. After the pulse (˜2 min) ends, switch the positive terminal to the next row and repeat steps 7 to 10. These steps need to be repeated until cells in all the 4 rows are subjected to electroporation.
STEP 12. Disconnect the electrical connections, lift the top plate, transfer all the LEPDs into a new transparent well plate.
STEP 13. Carefully pipette out the electroporation buffer from all the devices and add fresh cell culture media to the LEPDs and transfer them into the incubator.
STEP 14. This protocol for optimizing plasmid concentration along the columns and pulse parameters along the rows and can be repeated for as many combinations as desired.

Automated Imaging: an example process is illustrated and includes the following:
STEP 1. To clean the bottom of the LEPD membrane, which comes in contact with the delivery reagents, prepare a 12-well plate by dispensing 1 mL of PBS in each well, dip the LEPDs in the wells without fully submerging the devices (3 wells sequentially), and transfer the clean LEPDs to a transparent 24-well plate for imaging.
STEP 2. To quantify cell viability and delivery efficiencies, prepare a solution of Hoechst in PBS (0.1 mg/ml), gently aspirate the fluid from each well (cell media or electroporation buffer) without completely drying the well, and place 100 μl of Hoechst solution in the well for 10 min.
STEP 3. Gently aspirate the Hoechst solution, wash thrice with PBS, and dispense 150 μl of PBS for imaging.
STEP 4. Place the well plate containing the LEPDs in a motorized microscope stage.
STEP 5. Program the stage to move to the center of each well in the plate and use an objective lens with 10× or 20× magnification to capture images of the stained nuclei using a DAPI filter.
STEP 6. Focus on the cells manually using the course and fine focus knobs, or by programming the microscope with an autofocus routine. Briefly, the autofocus routine consists of calculating the focus score (e.g., normalized variance, Laplacian, or log-histogram) for a Z-stack of images acquired in a single field-of-view, moving to the Z-plane with the highest score, and iteratively reducing the step size and scanning range until achieving optimal focus. To reduce the time of the routine, the user manually focuses on the first well to set a reference Z-plane to be used for the subsequent wells since the difference in focus is small between wells.
STEP 7. Obtain images with multiple fluorescence and brightfield filters for each field-of-view. The choice of filters depends on the excitation and emission characteristics of the fluorescent probe to be examined.
STEP 8. For each LEPD acquire images at multiple fields-of-view by moving manually, or by programming the microscope to move to different locations within each well.
STEP 9: The representative transfection images of adherent and suspended cell lines, HeLa and K562, after plasmid (pmax GFP) delivery are presented in FIGS. 9A and 9B. The transfection efficiency appears to be lower in K562 cells, likely due to their non-adherent nature, which hampers efficient contact with the porous cell membrane. FIGS. 10A and 10B provide the viabilities images taken 24 hours post-transfection for HeLa and HEK 293T cell lines.

Image Segmentation and Data Analysis: an example process is illustrated and includes the following:
STEP 1. To identify the cells in the images, a fully convolutional network (FCN) (Long, Shelhamer et al. 2015) with a U-Net architecture (Falk, Mai et al. 2019) was trained using images and corresponding labels of various cell types and imaging modalities (e.g., fluorescence and phase-contrast). The FCN network consists of an encoder-decoder architecture containing 20 hidden layers (e.g., convolution, pooling, and up-convolution) that enables the classification of objects in the images (e.g., cell exterior, nucleus) with pixel-level resolution (Patino, Mukherjee et al. 2021, Mukherjee, Patino et al. 2022).
STEP 2. To train the U-Net for a specific cell type of interest, images of cells were manually labeled using drawing tools in an image manipulation software. To facilitate labeling of the cell exterior or internal cell compartments of interest, the cells were stained with fluorescent dyes (e.g., Hoechst nuclear dye or Calcein AM cytosolic dye) and imaged as described herein.
STEP 3. The images and their corresponding ground truth labels were split into training, validation, and test sets to cross-validate the training process and prevent overfitting.
STEP 4. A weighted soft-max cross entropy loss function was used to classify each pixel into three categories (interior, exterior, and boundary).
STEP 5. The U-Net was optimized using stochastic gradient descent (momentum=0.9, learning-rate=1×10⁻⁴). Training was performed using a graphic processing unit (GPU: NVIDIA) to expedite the training process for a large network containing more than 7.5 million trainable parameters.
STEP 6. To assess the performance of the model, the area overlap between the predicted objects and the ground truth labels was measured and used to determine true positives (TP), false positives (FP), true negatives (TN), and false negatives (FN). From these values the precision, recall and F1 scores could be calculated as follows:

$$precision = TP / ( TP + FP ) , recall = TP / ( TP + FN ) , \\ F 1 = 2 * ( precision * recall ) / ( precision + recall ) .$$

7. For analysis of viability, delivery, and transfection efficiencies the Hoechst nuclear images were used to segment to nuclei of the cells to determine the number of cells in each image. Moreover, the segmented nuclei were used as a mask to measure the fluorescence intensity of the other color channels (e.g., GFP filter for eGFP transfection and TexasRed filter for quantification of dead cells using propidium iodide). The fluorescence intensity for the respective filters was compared to negative control samples to determine the threshold for a positive signal. Viability and transfection efficiency were calculated as follows:

$$viability = 1 - ( {N}_{dead} / {N}_{cells} ) ; efficiency = {N}_{transfected} / {N}_{cells}$$

STEP 8. To extract measurements from the segmented images, a cell analysis software (e.g., CellProfiler) was used to obtain numerous features (e.g., shape, intensity, texture) from each cell. FIGS. 9C and 9D illustrate the efficiencies of GFP plasmid delivery into HEK 293T cells under varying voltages and pulse durations, and across different buffer conditions. The post-transfection viability results of HEK 293T cells with different voltages are shown in FIG. 10C where viability remains high between 10 to 20 V and declines at higher voltages of 30 and 40 V in both EP and DMEM buffer. FIG. 10D represents the viability results for HeLa, HEK and K562 cell lines post-transfection.
STEP 9. The features were transformed using the generalized logarithm (glog) method and standardized using the median and median absolute deviation (MAD) to obtain the robust Z score (R.Z. score). In contrast to the Z-score, the R.Z. score is not sensitive to outliers.
STEP 10. To analyze the features, correlation matrices and 2D feature projection maps (U-Map, t-SNE) can be applied.

Various features and advantages are set forth in the following claims.

What is claimed is:

1. An electroporation device comprising:
a first circuit board including a first plurality of electrodes;
a plurality of wells;
a bottomless well plate, each of the plurality of wells at least partially received within the bottomless well plate;
a second circuit board including a second plurality of electrodes;
wherein the bottomless well plate is positioned between the first circuit board and the second circuit board; and
wherein each of the first plurality of electrodes is at least partially positioned within one of the plurality of wells.

2. The electroporation device of claim 1, wherein the first plurality of electrodes is a plurality of pins.

3. The electroporation device of claim 2, wherein the plurality of pins are gold.

4. The electroporation device of claim 1, further comprising a spacer plate positioned between the bottomless well plate and the first circuit board.

5. The electroporation device of claim 1, wherein each of the plurality of wells includes a glass cylinder and a porous membrane.

6. The electroporation device of claim 1, wherein the second plurality of electrodes are electrically coupled to the first plurality of electrodes; wherein the electroporation device is configured to apply an electrical pulse to one of the plurality of wells.

7. A system comprising:
an electroporation device containing a plurality of cells;
a voltage generator electrically coupled to the electroporation device;
an optical microscope configured to capture an image of the plurality of cells; and
a computer including a processor and a non-transitory computer readable memory;
wherein the computer analyzes the image based on a machine learning model.

8. The system of claim 7, wherein the machine learning model extracts measurements of the plurality of cells from the image.

9. The system of claim 7, further comprising a multimeter electrically coupled to the electroporation device.

10. The system of claim 7, further comprising an incubator.

11. The system of claim 7, wherein the electroporation device includes 96 wells.

12. A method comprising:
culturing cells in an electroporation device;
applying a voltage to the electroporation device to deliver a cargo; and
analyzing cells post-electroporation.

13. The method of claim 12, wherein analyzing cells post-electroporation includes imaging, scRNAseq, mass spectroscopy, or any combination thereof.

14. The method of claim 12, wherein culturing cells further includes monitoring cells with live cell imaging.

15. The method of claim 12, wherein analyzing cells post-electroporation includes identifying differences in cell physiology and signal with a machine learning model.

16. The method of claim 12, wherein the cargo is nucleic acids, oligonucleotides, plasmid DNA, proteins, protein-spherical-nucleic-acids (pro-SNAs), siRNA, or Cas9ribonucleoprotein complexes (RNP).
